(19) United States
(12) Patent Application Publication
(10) Pub. No.: US 2023/0375605 A1
(43) Pub. Date: Nov. 23, 2023

(54) ACTIVE OPTICAL ELEMENTS BASED ON CHARGE DENSITY WAVE AND BROKEN SYMMETRY

(71) Applicant: UCHICAGO ARGONNE, LLC, Chicago, IL (US)

(72) Inventors: Pierre T. Darancet, Downers Grove, IL (US); Cristian L. Cortes, Chicago, IL (US); Stephen K. Gray, Wheaton, IL (US); Richard D. Schaller, Clarendon Hills, IL (US); Sahar Sharifzadeheh, Newport Coast, CA (US); Anubhab Haldar, Chicago, IL (US)

(21) Appl. No.: 18/198,026

(22) Filed: May 16, 2023

Related U.S. Application Data
(60) Provisional application No. 63/343,335, filed on May 18, 2022.

(51) Int. Cl. G01R 27/26

(52) U.S. Cl. CPC G01R 27/2682

(57) ABSTRACT

A method for identifying sufficient non-linear susceptibility in a test material. The method includes determining the polarizability of the test material, extracting from the polarizability, an optomechanical coupling of the test material, modeling light-induced dynamics, based on optomechanical coupling of the test material, and controlling the light induced dynamics to identify sufficient non-linear susceptibility.

### CROSS-REFERENCE TO RELATED APPLICATIONS

This application claims priority to and the benefit of U.S. Provisional Patent App. No. 63/343,335, filed May 18, 2022, the content of which is incorporated by reference in its entirety

### STATEMENT OF GOVERNMENT SUPPORT

This invention was made with government support under Contract No. DE-ACO2-06CH11357 awarded by the United States Department of Energy to UChicago Argonne, LLC, operator of Argonne National Laboratory. The government has certain rights in the invention.

### TECHNICAL FIELD

The present disclosure relates generally to active optical elements based on charge density wave and broken symmetry materials.

### BACKGROUND

Picosecond-scale light-induced dynamics are key in the preparation of non-thermal and transient phases with properties desirable for non-linear optics applications. Presently, layered transition metal dichalcogenides (“TMDC”) and their heterostructures, having strong coupling with light, are used in the search for novel transient electronic properties.

Experimental observations of the ultrafast laser-induced dynamics in the low-temperature, broken-symmetry phases of the layered TMDC 1T-TaS₂ have shown a strong coupling between light and structural distortions. A defining feature of 1T-TaS₂ is the strongly non-thermal response of the material, characterized by a selective, coherent excitation of the amplitude (Higgs) mode that displaces the system from its low-symmetry phase to its high-symmetry counterpart. The excitation of the Higgs mode has been observed using different techniques, including time-resolved absorption/reflection, ultrafast electron diffraction and microscopy, and time-resolved photoemission spectroscopy for excitation energies varying from 0.62 eV to 3.2 eV, suggesting a general, non-resonant coupling, and is also reproduced by first-principles calculations. Thus, 1T-TaS₂ is a prototypical material for study of broken-symmetry non-equilibrium phases of matter.

Currently, there is no microscopic mechanism that explains the coupling of structural order parameters and light in 1T-TaS₂, unlike other materials such as multiferroics. As 1T-TaS₂ is one of many known charge density wave (“CDW”) materials, this lack of a microscopic description also raises the question of the universality of the light-induced dynamics observed in 1T-TaS₂ for this class of materials.

### SUMMARY

Embodiments described herein relate generally to a method for identifying sufficient non-linear susceptibility in a test material. The method includes determining a polarizability of the test material, extracting from the polarizability, an optomechanical coupling of the test material, modeling light-induced dynamics, based on the optomechanical coupling of the test material, and controlling the light-induced dynamics to identify sufficient non-linear susceptibility.

It should be appreciated that all combinations of the foregoing concepts and additional concepts discussed in greater detail below (provided such concepts are not mutually inconsistent) are contemplated as being part of the subject matter disclosed herein. In particular, all combinations of claimed subject matter appearing at the end of this disclosure are contemplated as being part of the subject matter disclosed herein.

### BRIEF DESCRIPTION OF DRAWINGS

The foregoing and other features of the present disclosure will become more fully apparent from the following description and appended claims, taken in conjunction with the accompanying drawings. Understanding that these drawings depict only several implementations in accordance with the disclosure and are not, therefore, to be considered limiting of its scope, the disclosure will be described with additional specificity and detail through use of the accompanying drawings, in which:

FIG. 1A shows the total energy of 1T-TaS₂ with respect to the Higgs and Goldstone coordinates computed using quantum mechanical calculations;

FIGS. 1B and 1C show corresponding real parts of the in-plane dielectric function (∈_{∥}) at two different excitation wavelengths computed using quantum mechanical calculations;

FIG. 2A shows the Raman cross-section with the amplitude mode for incoming light;

FIG. 2B shows real and imaginary parts of the Raman tensor at (r_{eq}, θ=0) of the amplitude mode as a function of light frequency;

FIG. 2C is a comparison of 1T-TaS₂ Raman with other materials;

FIG. 3A shows time-evolution of the amplitude mode after excitation by short optical pulses of wavelengths, a time profile of the pulse, and a Fourier transform of the damped response after the initial transient response has subsided; and

FIG. 3B shows Poincaré surfaces of sections at different energies of the conjugate variables r and r when crossing the \dot{θ}=0 plane for the pure potential demonstrating the existence of regular orbits.

Reference is made to the accompanying drawings throughout the following detailed description. In the drawings, similar symbols typically identify similar components, unless context dictates otherwise. The illustrative implementations described in the detailed description, drawings, and claims are not meant to be limiting. Other implementations may be utilized, and other changes, such as changes involving other broken symmetry materials and excitation protocols, may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

### DETAILED DESCRIPTION OF VARIOUS EMBODIMENTS

Before turning to the figures, which illustrate certain exemplary embodiments in detail, it should be understood that the present disclosure is not limited to the details of methodology set forth in the description or illustrated in the figures. It should also be understood that the terminology used herein is for the purpose of description only and should not be regarded as limiting.

As utilized herein, the term “1T-TaS₂” and the like refers to Tantalum Disulfide in the 1T phase. The 1T phase refers to the octahedral phase of Tantalum Disulfide.

Generally, while non-linearity is known to be desirable for materials in certain field so fuse, such as quantum sensing and imaging, there remains a need for a general descriptor for such materials and a method for identifying materials having such properties.

The present disclosure relates to a method for identifying materials with sufficient non-linear susceptibility. In some embodiments, sufficient non-linear susceptibility may be non-linear susceptibility that is higher than what is currently known in the art. More specifically, the method utilizes the coupling between light and the structural order parameter in materials with a broken symmetry structural ground state, such as the charge density wave state of the layered transition-metal dichalcogenide, tantalum disulfide (1T-TaS₂).

In some embodiments, materials having broken symmetry ground-state with a dielectric contrast with the high symmetry phase, irrespective of the stability of the high symmetry phase, including rare earth nickelates (e.g., anion containing nickel, salt containing a nickelate anion, double compound containing nickel bound to oxygen and other elements, etc.), charge density wave materials, or other materials having broken symmetry ground-states may be used as the test material, in addition to or replacing 1T-TaS₂. Using time-dependent density functional theory calculations of the dielectric properties along the distortions coordinates, 1T-TaS₂ displays a change in its dielectric function along the amplitude (Higgs) mode. This change originates from the coupling of the periodic lattice distortion with an in-plane metal-insulator transition, leading to optomechanical coupling coefficients two orders of magnitude larger than the ones of diamond and ErFeO₃. In addition, an effective model of the light-induced dynamics is derived, which is in quantitative agreement with experimental observations in 1T-TaS₂. Light-induced dynamics along the structural order parameter in 1T-TaS₂ may be deterministically controlled to engineer large third-order non-linear optical susceptibilities. The methods discussed herein suggest that CDW materials are promising active materials for non-linear optics.

Using calculations, such as density functional theory (“DFT”) and time-dependent density functional theory (“TDDFT”), the frequency-dependent polarizability of 1T-TaS₂ along its structural Higgs and Goldstone coordinates is determined. The coupling of lattice distortions with a metal-insulator transition leads to large (e.g., larger than previous results) optomechanical coupling coefficients, at least two orders of magnitude larger than the ones of diamond, BiFeO₃, and ErFeO₃. Using TDDFT results, an effective classical model of the light-induced dynamics of the structural Higgs and Goldstone modes in CDW materials is derived and in quantitative agreement with experimental observations of light-induced dynamics in 1T-TaS₂. These effects enable the light-induced dynamics of the Higgs mode to be deterministically controlled, in order to engineer large non-linear optical susceptibilities in materials that couple periodic lattice distortions with metal-insulator transitions. Such materials are promising active materials for non-linear optics. Using the described method to 1T-TaS₂, the magnitude and frequency of the third order non-linear susceptibility is directly controlled by the polarization/frequency/and pulse profile dependence of the driving pulse(s). Depending on the chosen driving pulse excitation protocol and the resulting regular orbit along the Goldstone and Higgs coordinate, the third order non-linear susceptibility in 1T-TaS₂ can be tuned from negligible to exceeding the ones of the ones of diamond, BiFeO₃, and ErFeO₃ by four orders of magnitude.

In some embodiments, the methods and processes described herein may be facilitated by a computing system, the computing system including, at least, a processor and a memory. The memory including machine-readable instructions, that when executed, cause the processors to complete the methods and processes described herein. In some embodiments, the computational methods described herein may be measured, observed, determined, or the like by a sensor (e.g., probe, system, etc.). The sensor may be communicatively coupled to the computing system and configured to send and receive signals, data, and/or information to and from the computing system.

The hardware and data processing components used to implement the various processes, operations, illustrative logics, logical blocks, modules and circuits described in connection with the embodiments disclosed herein may be implemented or performed with a general purpose single- or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some embodiments, particular processes and methods may be performed by circuitry that is specific to a given function. The memory (e.g., memory, memory unit, storage device) may include one or more devices (e.g., RAM, ROM, Flash memory, hard disk storage) for storing data and/or computer code for completing or facilitating the various processes, layers and modules described in the present disclosure. The memory may be or include volatile memory or non-volatile memory, and may include database components, object code components, script components, or any other type of information structure for supporting the various activities and information structures described in the present disclosure. According to an exemplary embodiment, the memory is communicably connected to the processor via a processing circuit and includes computer code for executing (e.g., by the processing circuit or the processor) the one or more processes described herein.

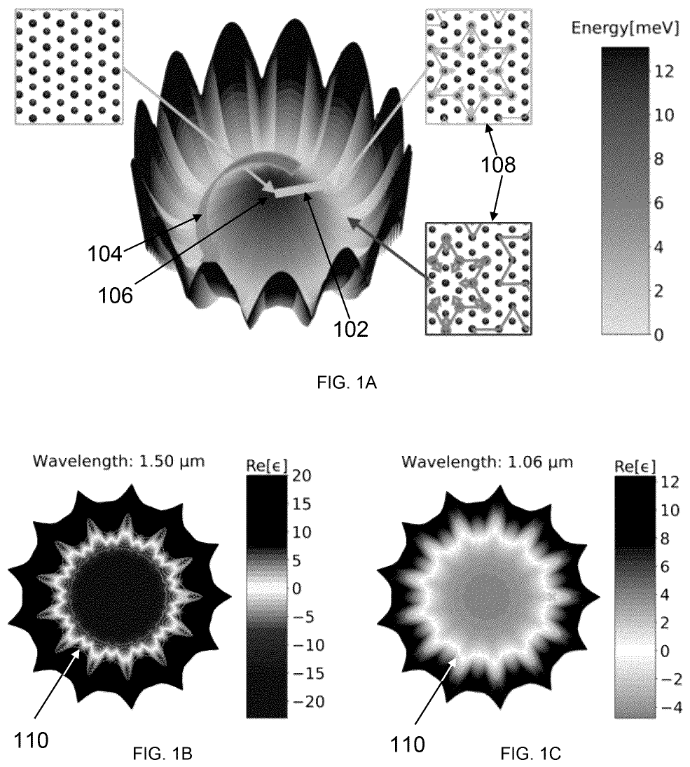

FIG. 1A illustrates a determined total energy of 1T-TaS₂ with respect to the Higgs and Goldstone coordinates. The Higgs and Goldstone coordinates correspond respectively to the oscillations of the amplitude 102 and phase mode 104, respectively. Where the amplitude 102 is defined radially away from a center 106 and the phase mode 104 is defined about the center 106. In some embodiments, such as the embodiment of FIG. 1A, the range of displacement along the radial coordinate is 1.30 Å. The energy is indicated per TaS₂. The minima along the Goldstone directions correspond to a Tantalum atom at the center of the displacement field.

In the context of periodic lattice distortions, the Higgs coordinate corresponds to the amplitude of the vector field describing atomic displacements between the high symmetry (1 Ta atom per unit cell) and low symmetry (13 Ta atom per unit cell) configurations. Correspondingly, the Goldstone coordinate refers to the origin of this vector field within the unit cell. As solids lack continuous symmetry, energy fluctuates along the Goldstone coordinate. In 1T-TaS₂, the ground-state CDW configuration is associated with a Ta atom at the center of the distortion, hence the energy has 13 distinct minima along the Goldstone coordinate. As is customary, the Higgs coordinate is represented by the radial r direction and the Goldstone coordinate is represented by the angular θ direction.

At temperatures lower than 180° K, 1T-TaS₂, along with the isovalent 1T-TaS₂, exhibits, as seen in star views 108, a so-called “Star-of-David” commensurate CDW involving an in-plane, \sqrt{13}×\sqrt{13}R=12.4° periodic-lattice distortion, associated with an insulating state—unlike the metallic behavior of the high-temperature undistorted phase. At this low temperature, the nature of the low-temperature insulating state may possibly be that of a Mott insulator or an in-plane band insulator with Anderson localization out-of-plane caused by packing disorder. While Mott insulators and the aforementioned in-plane band insulators may differ in out-of-plane character, both are compatible with the DFT description, described below, of the in-plane insulating band structure. Such compatibility is in agreement with angle-resolved photoemission spectroscopy, vibrational spectroscopy, and time-resolved spectroscopy experiments. All configurations of 1T-TaS₂ are modeled using a \sqrt{13}×\sqrt{13}×1 unit cell with 13 Tantalum atoms, and a vertical stacking of center of distortions. Such a configuration is used as the unit cell is conducive to in-plane insulating band structures.

The total energy, as shown in FIG. 1A, as well as the components of the macroscopic longitudinal dielectric function tensor, ∈(q→0; ω), as later discussed in reference to FIGS. 1B and 1C, is computed using a projector-augmented wave method (GPAW) implementations of DFT and TDDFT, wherein the GPAW implement of DFT is used to compute the total energy and the GPAW implementation of TDDFT is used to compute the components of the tensor. A generalized gradient approximation of Perdew, Burke, and Ernzerhof (“PBE”) with an effective Hubbard U value of 2.27 eV, with 5 (6) valence electrons is used for Ta (S). TDDFT calculations are performed within the random phase approximation (“RPA”). An inhomogeneous configuration grid is used for interpolations of the total energy and dielectric functions. In some embodiments, such as the embodiment used in FIGS. 1A-1C, the inhomogeneous configuration gird includes 188 configurations.

FIG. 1A shows the interpolated DFT total energy along the Higgs and Goldstone coordinates. The extrema of the total energy include the 13 distinct energy minima at r_{eq}≈0.77 Å and θ≡0 mod 2π/13, with a local maximum associated with the high symmetry cell (r=0) 12.7 meV/TaS₂. Additionally, saddle points of energy 2.65 meV/TaS₂ are found at (r=0.65 Å, θ≡π/13 mod 2π/13) using the nudged elastic band method, confirming that the Goldstone mode acquires a finite energy due to the discrete lattice symmetry.

FIGS. 1B and 1C illustrate corresponding real parts of the in-plane dielectric function (∈_{∥}) at two different excitation wavelengths. FIG. 1B illustrates the real parts of the in-plane dielectric function at a wavelength of 1.50 μm. FIG. 1C illustrates the real parts of the in-plane dielectric function at a wavelength of 1.06 μm. In both FIGS. 1B and 1C, a plurality of isoenergy contours 110 are placed 0.77 meV/TaS₂ apart, starting at 0.77 meV.

In 1T-TaS₂, the periodic lattice distortion is associated with an in-plane metal-insulator transition. Accordingly, and as shown in FIG. 1B, the real part of the dielectric function Re [∈(ω; r, θ)] of 1T-TaS₂ is negative near the high symmetry cell, r=0, and becomes positive at values of r near the ground state (r_{eq}≈0.77 Å). As seen when comparing FIGS. 1B and 1C, the r value at which the transition occurs Re [∈(ω; r, 0)]=0 increases as co increases. Moreover, the amplitude of the change along the Higgs coordinate, |Re[∈(ω; 0, 0)−∈(ω; r_{eq}, 0)]|, decreases as co increases. This decrease is a consequence of higher energy transitions having a weaker dependence on the atomic details of the lattice due to their higher kinetic energy.

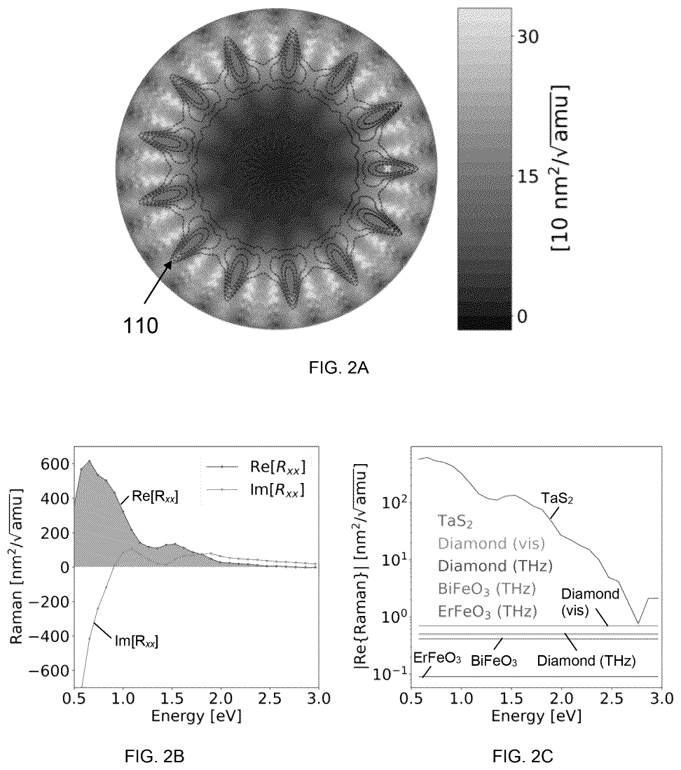

Importantly, for all r, the relative variations of Re[∈(ω; r, θ)] along r are large (e.g., with values |Re[∈(ω; 0, 0)−∈(ω; r_{eq}, 0)]/Re[∈(ω; r_{eq}, 0)]|>>1). In contrast, the variations of Re[∈(ω; r, θ)] along θ, while finite, are found to be on the order of Re[∈(ω; r, θ=π/13)]−Re[∈(ω; r, θ=0)]<5. This variation of ∈(ω; r, θ) along the Higgs coordinate has an impact on the Raman activity (i.e., the Raman tensor) and is related to the optomechanical coupling coefficient which is further described in reference to FIG. 2A.

FIG. 2A illustrates the Raman cross-section with the amplitude mode for incoming light. In some embodiments, such as the embodiments of FIG. 2A,

$$ω = 1.17 eV \frac{\partial α ( ω ; r , θ )}{\partial r} ( r , θ ) / \sqrt{m} .$$

A plurality of isoenergy contours 110 are placed 0.77 meV/TaS₂ apart, starting at 0.77 meV.

The optomechanical coupling coefficient can be extracted from direct differentiation of the RPA polarizability α(ω; r, θ), computed using TDDFT, along the distortion coordinates, as shown in FIG. 2A. The optomechanical coupling between light and the Higgs order parameter,

$${m}^{- 1 / 2} \frac{\partial α ( ω ; r , θ )}{\partial r} ( r , θ )$$

(where m is the effective mass of the mode), is maximal near the CDW ground-state with r=r_{eq}, progressively vanishing near the high symmetry phase as r→0. Thus, in 1T-TaS₂, light can only transiently stabilize the high-symmetry phase, and that the amplitude mode cannot be excited from the high-symmetry phase. Moreover, the optomechanical coupling near r_{eq} depends on θ more significantly than at other values of r, suggesting that a protocol of excitation involving the Goldstone mode could further enhance the optomechanical coupling.

FIG. 2B illustrates real and imaginary parts of the Raman tensor at (r_{eq}, θ=0) of the amplitude mode as a function of light frequency. As seen in the FIG. 2B, the optomechanical coupling at (r=r_{eq}, θ=0) decreases at higher frequencies, this corresponds with the larger dielectric changes at lower frequencies, as seen in FIGS. 1A-1C. The magnitude of the imaginary part of the Raman tensors at ω≲1.0 eV increases, as co decreases, quicker than when ω>1 eV. This behavior of the imagine part suggests that 1T-TaS₂ could be used as a platform for entangled photon emission, as the imaginary part is related to the two-photon absorption/emission of the materials.

FIG. 2C illustrates a comparison of 1T-TaS₂ Raman with other materials. As seen in FIG. 2C, the large change in dielectric function (ΔRe[ϵ]≈50) over a small displacement (Δr<4 Å) enabled by the charge density wave results in a value of the optomechanical coupling coefficient that is two orders of magnitude greater than reference materials, such as BiFeO₃, ErFeO₃ and diamond. The values for BiFeO₃, ErFeO₃ and diamond are taken from JURASCHEK & MAEHRLEIN, “Sum-frequency ionic Raman scattering,” Physical Review B 97(17):174302, 8 pages (2018).

The large (e.g., greater than reference materials) optomechanical coupling in a CDW material is significant when searching for materials with large third-order non-linear susceptibility x⁽³⁾(ω₁, ω₂, ω₃). In particular, x⁽³⁾(ω₁, ω₂, ω₃)∝|Rω₁|²H(ω₂, ω₃), where |R|² is the square of the Raman tensor plotted in FIGS. 2A-2C, and H(ω, ω′) is the phonon propagator. As seen in FIG. 2C, the large (e.g., greater than reference materials) values of |R| suggest that 1T-TaS₂ and other CDW materials could be promising for non-linear optics applications.

However, to use CDW materials for non-linear optics, a deterministic trajectories of the system with light, across configurations with large dielectric constant contrast must be generated. To analyze the possibility of CDW materials being used for non-linear optics, a classical equation of motion is used, describing the Higgs and Goldstone coordinates coupled with light through Raman scattering derived in:

$$m \ddot{X} = - \frac{\partial U}{\partial X} - γ \dot{X} + R {E}^{2} \qquad ( Eq . 1 )$$

where X=(r, θ), U is the total energy computed in DFT, γ is an empirical damping parameter, and E² the electric field associated with an in-plane polarization. The forces and Raman tensor are computed and interpolated from DFT and TDDFT, respectively, at each (r, θ). A classic description based on the Born-Oppenheimer approximation is not suitable for the description of the sub-picosecond dynamics and describes the system evolution at times larger than the timescales associated with electronic thermalization.

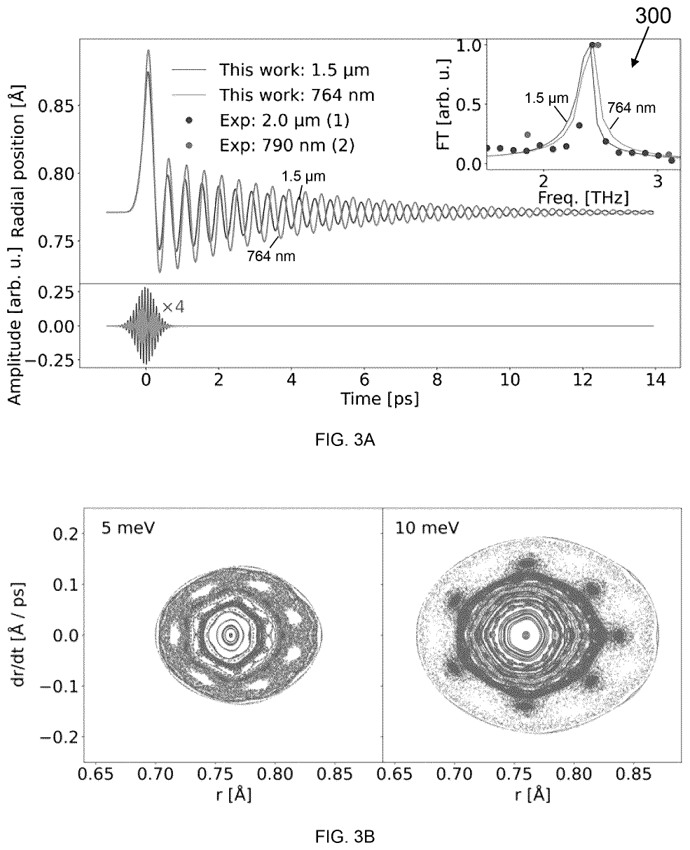

FIG. 3A illustrates time-evolution of the amplitude mode after excitation by short optical pulses of wavelengths predicted by Eq. 1 with an empirical damping parameter γ=0.5 THz, a time profile of the pulse. In some embodiments, the excitation is driven by a resonant terahertz probe. Insert 300 illustrates a Fourier transform of the damped response after the initial transient response has subsided and compares to experimental data from (1) DEAN, et al., “Polaronic Conductivity in the Photoinduced Phase of T-TaS₂,” Physical Review Letters 106(1):016301, 4 pages (2011) and (2) from HELLMAN, et al. “Time-domain classification of charge-density-wave insulators,” Nature Communications 3:1069, 8 pages (2012).

As the Raman tensors in Eq. 1 depends on the frequency of the pulse, ω, the coupling between light intensity and dynamics is also explicitly dependent on ω, with lower values of ω resulting in stronger coupling at constant intensity. In some embodiments, as in FIG. 3A, the time-evolution is computed using a pulse intensity of 5×10⁻³ V/Å and 2×10⁻² V/Å for the infrared and visible pulse respectively. The predicted time-evolution at these pulse intensity values is in quantitative agreement with the experimental values from (1) and (2), validating the non-resonant coupling mechanism CDW order parameter and light through Raman processes as the mechanism underlying light-induced dynamics in 1T-TaS₂. Furthermore, the low energy of the saddle points ((≈35 meV per unit cell of 13 Ta atoms) along the Goldstone coordinate suggests a possible chaotic dynamics even at low pulse energy.

FIG. 3B illustrates Poincaré surfaces of sections at different energies (Left: 5 meV/13Ta, Right: 10 meV/13Ta) of the conjugate variables r and \dot{r} (corresponding to the movement and position along the Higgs mode) when crossing the θ=0 plane for the pure potential (no damping nor external field). Poincare surfaces of sections are constructed using orbits generated from Eq. 1 without dissipation or coupling with light to test the stability of the dynamics necessary to generate large non-linear susceptibility through optomechanical coupling. At excess energies as low as 5 meV and 10 meV per unit cell, as in FIG. 3B, the phase space displays some regions associated with chaotic trajectories and regular trajectories separated by Kolmogorov-Arnold-Moser tori. The chaotic regions are associated with large fluctuations in r (i.e., small deviations in the θ directions). Regular orbits exist for lower fluctuations in r around r_{eq}, on the scale of r_{eq}±0.05 Å. These results suggest that excitation of the system along the Goldstone coordinate is needed to generate regular orbits with Higgs fluctuations. At higher energies, the phase space is still mixed, with the areas of chaotic and regular orbits both expanding. This indicates that an initial excitation protocol of the system along both Higgs and Goldstone coordinates may be necessary for harnessing the optomechanical effect in x⁽³⁾ applications.

The present disclosure demonstrates a method for identifying materials with high non-linear susceptibility. Using DFT/TDDFT calculations, the optomechanical coupling of 1T-TaS₂ was determined to be larger than other known materials, due to the coupling between the periodic lattice distortion and a metal-insulator transition. The calculations were in quantitative agreement with time-resolved experiments. The results demonstrated that the coupling between light and the structural order parameter in the CDW state of 1T-TaS₂ is mediated by the change in dielectric function along the amplitude (Higgs mode), suggesting that CDW materials are promising active media for non-linear optics applications, provided the system is carefully prepared in a mixture of Higgs and Goldstone modes.

As used herein, the singular forms “a,” “an,” and “the” include plural referents unless the context clearly dictates otherwise. Thus, for example, the term “a member” is intended to mean a single member or a combination of members, “a material” is intended to mean one or more materials, or a combination thereof.

As used herein, the terms “about” and “approximately” generally mean plus or minus 10% of the stated value. For example, about 0.5 would include 0.45 and 0.55, about 10 would include 9 to 11, about 1000 would include 900 to 1100.

It should be noted that the terms “example”, “exemplary”, or the like as used herein to describe various embodiments are intended to indicate that such embodiments are possible examples, representations, and/or illustrations of possible embodiments (and such term is not intended to connote that such embodiments are necessarily extraordinary or superlative examples).

The terms “coupled,” “connected,” and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

It is important to note that the construction and arrangement of the various exemplary embodiments are illustrative only. Although only a few embodiments have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. Other substitutions, modifications, changes and omissions may also be made in the design, operating conditions and arrangement of the various exemplary embodiments without departing from the scope of the present invention.

While this specification contains many specific implementation details, these should not be construed as limitations on the scope of any inventions or of what may be claimed, but rather as descriptions of features specific to particular implementations of particular inventions. Certain features described in this specification in the context of separate implementations can also be implemented in combination in a single implementation. Conversely, various features described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

What is claimed is:

1. A method for identifying sufficient non-linear susceptibility in a test material, the method comprising:
determining a polarizability of the test material;
extracting from the polarizability, an optomechanical coupling of the test material;
modeling light-induced dynamics, based on the optomechanical coupling of the test material; and
controlling the light-induced dynamics to identify sufficient non-linear susceptibility.

2. The method of claim 1, the method further comprising:
determining a dielectric tensor of the test material,
wherein the polarizability and the dielectric tensor are determined along Higgs and Goldstone coordinates of the test material using a time-dependent density functional theory.

3. The method of claim 1, wherein the light-induced dynamics along Higgs and Goldstone coordinates are determined using a mixed classical-quantum framework.

4. The method of claim 1, the method further comprising:
determining a total energy of the test material using density functional theory.

5. The method of claim 1, wherein the test material is a material having a structural ground-state of broken symmetry.

6. The method of claim 5, wherein the test material is a material that exhibits dielectric contrast between its high symmetry phase and broken symmetry ground state.

7. The method of claim 5, wherein the test material is a material that exhibits charge density waves.

8. The method of claim 5, wherein the test material is a material that exhibits a metal-insulator transition coupled to its structural distortion.

9. The method of claim 5, wherein the test material is a material that exhibits a semimetal to metal transition coupled to its structural distortion.

10. The method of claim 1, the method further comprising:
determining regular orbits by testing along Higgs and Goldstone structural coordinates in the light-induced dynamics of the test material.

11. The method of claim 10, the method further comprising:
analyzing susceptibility fluctuations along the regular orbits generated by the optomechanical coupling.
